(19) United States
(12) Patent Application Publication
(10) Pub. No.: US 2022/0370650 A1
(43) Pub. Date: Nov. 24, 2022

(54) FORMULATIONS AND KITS FOR THE DIAGNOSTIC EVALUATION OF INFECTIOUS DISEASES AND METHODS THEREOF

(71) Applicant: Vyripharm Enterprises, Inc., Houston, TX (US)

(72) Inventor: Jerry L. BRYANT, JR., Bellaire, TX (US)

(21) Appl. No.: 17/697,290

(22) Filed: Mar. 17, 2022

Related U.S. Application Data
(60) Provisional application No. 63/166,898, filed on Mar. 26, 2021.
(60) Provisional application No. 63/166,904, filed on Mar. 26, 2021.
(60) Provisional application No. 63/166,910, filed on Mar. 26, 2021.
(60) Provisional application No. 63/166,866, filed on Mar. 26, 2021.
(60) Provisional application No. 63/166,877, filed on Mar. 26, 2021.

(51) Int. Cl. A61K 51/08; A61K 51/04

(52) U.S. Cl. CPC A61K 51/088; A61K 51/0491; A61K 51/0459; A61K 51/0455; A61K 2123/00

(57) ABSTRACT

Kits for the preparation, synthesis, and delivery of compositions comprising a conjugate of a nucleoside analog, a chelator, and a label for use as imaging and therapeutic agents. The kits, as well as methods for their use, may be used in diagnosing, treating, or monitoring the progression of infectious diseases and/or symptoms or complications resulting from infection.

### CROSS-REFERENCE TO RELATED APPLICATIONS

This application claims the benefit of priority to U.S. Provisional Application Ser. No. 63/166,898, filed Mar. 26, 2021, 63/166,904, filed Mar. 26, 2021, 63/166,910, filed Mar. 26, 2021, 63/166,866, filed Mar. 26, 2021, and 63/166,877, filed Mar. 26, 2021, the entire contents of which are hereby incorporated by reference, for all purposes, in their entirety.

### FIELD OF TECHNOLOGY

The present disclosure is directed to compositions, formulations, and kits for targeted infectious disease diagnosis and methods for the treatment of infectious diseases. The present disclosure is also related to methods for compounding drugs and drug compositions for use in kits and drug delivery systems.

### BACKGROUND

The diagnosis and treatment of many infectious diseases continues to be poorly understood. In particular, since the COVID-19 outbreak was declared a public health emergency of international concern by the World Health Organization (WHO) on Jan. 30, 2020, the progression of the severe acute respiratory syndrome coronavirus 2 (SARS-COV-2) virus has reminded us of the critical role of an effective host immune response as well as the devastating effect of immune dysregulation. Accordingly, there is a critical need for an efficient approach for both the diagnosis and treatment of infectious diseases, such as COVID-19. However, inconsistencies in the diagnosis and stage identification of infectious diseases and their progression persist making identification of appropriate and efficient treatment protocols challenging. Furthermore, effective approaches to tailored or targeted treatment of infectious diseases has also been challenging, including attempts at developing vaccines. Accordingly, there is a need for improved approaches for the accurate diagnosis as well as evaluation of disease progression and severity in order to provide patient-specific targeted treatment protocols and methodologies. There is also a need for improved methods of evaluating patient-specific treatment outcomes.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is exemplary process for the synthesis of the compound according to structural formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, according to an exemplary embodiment of the present disclosure; and

FIG. 2 is an exemplary process for the synthesis of the compound according to structural formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide], according to an exemplary embodiment of the present disclosure.

### DETAILED DESCRIPTION

It will be appreciated that numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

The present disclosure provides kits to prepare an imaging probe, a diagnostic agent, or a pharmaceutical composition. The present disclosure also provides methods of imaging, diagnosing, and delivering a pharmaceutical composition to treat infectious diseases with nucleoside analogs prepared using the disclosed kits. The presently disclosed methods and kits may be used to assess the personalized and efficacious dose and dosing regiments based on accurate evaluations determined through molecular imaging using nucleoside analog.

According to at least one aspect of the present disclosure, a kit for preparing an imaging probe composition suitable for administration to a subject in need thereof is provided. The imaging probe composition may include a conjugate of a nucleoside analog, a chelator, and a label. The kit may include a first sealed container that contains a predetermined quantity of a precursor composition. The precursor composition may include a conjugate of a nucleoside analog and a chelator. The kit may also include a second sealed container containing a predetermined quantity of a label composition. The label composition may comprise at least one imaging agent or at least one radionuclide label. In at least some instances, the radionuclide label may be a radionuclide metal ion. In at least some instances, the kit may also include a third sealed container comprising a predetermined quantity of a cannabinoid composition comprising one or more cannabinoid compounds. The sealed containers may be, for example, a bottle, vial, ampule, or syringe, or any other suitable container.

During use and prior to administration to the subject, the precursor composition may be contacted with the label composition to form the imaging probe composition. In cases in which the kit includes a third sealed container that includes a cannabinoid composition, the precursor composition may be contacted with cannabinoid composition to form a cannabinoid-chelator-nucleoside analog conjugate. In some instances, at least one of the first sealed container, the second sealed container, and the third sealed container may further include a predetermined quantity of a reducing agent. The reducing agent may have a predetermined quantity sufficient to label the conjugate of a nucleoside analog and a chelator with the radionuclide label or imaging agent to form the imaging probe composition. In at least some instances, the reducing agent may include tin (II) chloride.

In some instances, at least one of the first sealed container, the second sealed container, and the third sealed container may further include a buffer solution. In some cases, the first sealed container and the precursor composition contained therein includes a reducing agent and a buffer solution. The buffer solution may be, for example, a phosphate buffer solution. In such cases, the phosphate buffer solution may be present in sufficient quantity to stabilize the conjugate of a nucleoside analog and a chelator. The phosphate buffer solution may be, for instance, an aqueous solution comprising monosodium phosphate or disodium phosphate, or any combination thereof.

According to one aspect of the present disclosure, the first sealed container and the precursor composition may include an antioxidant. For example, the antioxidant may be present in sufficient quantity to prevent oxidation of the chelator moiety in the conjugate of a nucleoside analog and a chelator. The antioxidant may be vitamin C (ascorbic acid). The antioxidant may also be selected from the group consisting of tocopherol, pyridoxine, thiamine, butylated hydroxyl toluene, sodium edetate, rutin, vitamin C (ascorbic acid), and any combination thereof.

According to another aspect, the first sealed container and the precursor composition contained therein may include a stabilizer. For example, the stabilizer may be present in sufficient quantity to prevent degradation and enhance shelf life or storage life of the chelator moiety in the conjugate of a nucleoside analog and a chelator. The stabilizer may be mannitol or may be selected from the group consisting of glucose, lactose, maltose, xylose, sorbitol, cellulose, carboxymethylcellulose sodium, and any combination thereof. In other instances, the stabilizer may be a sugar or bulking agent. For example, the sugar may be selected from the group consisting of simple sugars, complex chain sugars, sugar alcohols, and any combination or salt thereof.

In other aspects, at least one of the first sealed container, the second sealed container, and the third sealed container, and/or the respective compositions contained therein may include a pharmaceutically acceptable salt or a preservative. The precursor composition, imaging agent, and/or labeling composition may be in any form, including but not limited to, liquid, frozen, dry, or lyophilized form.

The nucleoside analog may be a guanine analog. In other cases, the nucleoside analog may be a cell replication check point ligand. In some instances, the nucleoside analog may be a synthetic analog. In other instances, the nucleoside analog may be a natural analog. In some cases, the nucleoside analog may be guanine. According to at least one aspect, the nucleoside analog may be selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof. The nucleoside analog may, in some instances, be arabinosyl nucleoside.

In some instances, the chelator may be an aminated chelator or an acid chelator. In some instances, the chelator may be a N4 chelator or ligand. The chelator, may be, for example, cyclam, 6-carboxy-1,4,8,11-tetraazaundecane, or 1,4,8,11-tetraazabicyclohexadecane.

The radionuclide label may be selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof. In at least some instances, the radionuclide label may be configured to facilitate contrast-enhanced imaging when administered to a mammalian subject in conjunction with diagnostic imaging.

The conjugate in the precursor composition may be a N4-guanine (N4amG) such as cyclam-am-guanine. In some instances, the conjugate may comprise N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, corresponding to a compound characterized by the structure according to Formula

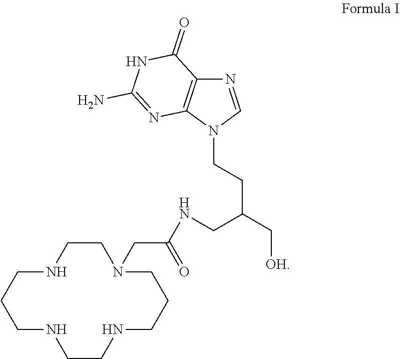 [SMILES: Nc1nc2c(ncn2CCC(CO)CNC(=O)CN2CCCNCCNCCCNCC2)c(=O)[nH]1]

In other instances, the conjugate may comprise N-(9-(4-amino-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula II:

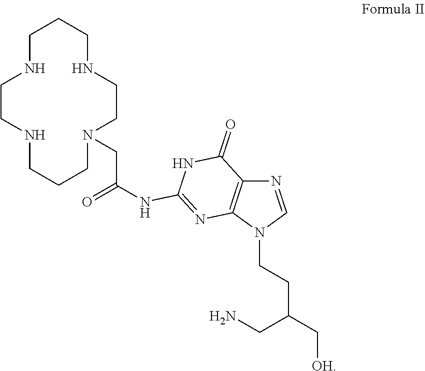 [SMILES: NCC(CO)CCn1cnc2c(=O)[nH]c(NC(=O)CN3CCCNCCNCCCNCC3)nc21]

In still other instances, the conjugate may comprise N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula III:

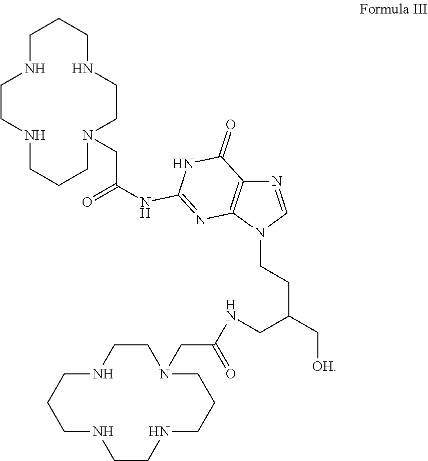 [SMILES: O=C(CN1CCCNCCNCCCNCC1)NCC(CO)CCn1cnc2c(=O)[nH]c(NC(=O)CN3CCCNCCNCCCNCC3)nc21]

In still other instances, the conjugate may comprise 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide], corresponding to a compound characterized by the structure according to Formula IV:

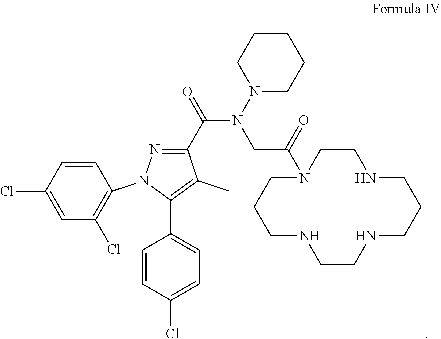 [SMILES: Cc1c(C(=O)N(CC(=O)N2CCCNCCNCCCNCC2)N2CCCCC2)nn(-c2ccc(Cl)cc2Cl)c1-c1ccc(Cl)cc1 ; "."]

In at least some instances, the conjugate of a nucleoside analog and a chelator comprises one or more cannabinoid compounds such that the conjugate of a nucleoside analog and a chelator is a cannabinoid-chelator-nucleoside analog conjugate. The one or more cannabinoid compounds used in the precursor composition or used in the third sealed container may include a synthetic or natural cannabinoid compound, or any combination thereof. The one or more cannabinoid compounds may comprise a cannabinoid receptor agonist or a cannabinoid receptor antagonist. In some instances, the cannabinoid receptor may be a cannabinoid receptor subtype CB₁ or a cannabinoid receptor subtype CB₂. In other cases, the cannabinoid receptor may be a non-CB₁ and a non-CB₂ receptor.

The one or more cannabinoid compounds may be a synthetic cannabinoid compound selected from the group consisting of N-(piperidin-1-yl)-5-(4-chlorophenyl)-1-(2,4-dichlorophenyl)-4-methyl-1H-pyrazole-3-carboxamidehydrochloride (SR141716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3 carboxamide (AM281), 4-[6-Methoxy-2-(4-methoxyphenyl)benzofuran-3-carbonyl]benzonitrile (LY320135), and any combination thereof. The one or more cannabinoid compounds may be a 5-(1,1-Dimethylheptyl)-2-[5-hydroxy-2-(3-hydroxypropyl)cyclohexyl]phenol (CP-55940), (6aR, 10aR)-6,6,9-trimethyl-3-pentyl-6a,7,8,10a-tetrahydrobenzo[c]chromen-1-ol (Dronabinol or Marinol), or (6aR,10aR)-1-hydroxy-6,6-dimethyl-3-(2-methyloctan-2-yl)-7,8,10,10a-tetrahydro-6aH-benzo[c]chromen-9-one (Nabilone).

In some instances, the one or more cannabinoid compounds may be a synthetic cannabinoid compound selected from the group consisting of diarylopyrazole, N-(piperidin-1-yl)-5-(4-chlorophenyl)-1-(2,4-dichlorophenyl)-4-methyl-1H-pyrazole-3-carboxamidehydrochloride (SR141716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3carboxamide (AM281), 4-[6-Methoxy-2-(4-methoxyphenyl)benzofuran-3-carbonyl]benzonitrile (LY320135), 5-(1,1-Dimethylheptyl)-2-[5-hydroxy-2-(3-hydroxypropyl)cyclohexyl]phenol (CP-55940), (6aR,10aR)-6,6,9-trimethyl-3-pentyl-6a,7,8,10a-tetrahydrobenzo[c]chromen-1-ol (Dronabinol or Marinol), (6aR,10aR)-1-hydroxy-6,6-dimethyl-3-(2-methyloctan-2-yl)-7,8,10, 10a-tetrahydro-6aH-benzo[c]chromen-9-one (Nabilone), an aminoalkylindole, (2-iodo-5-nitrophenyl)-(1-(1-methylpiperidin-2-ylmethyl)-1H-indol-3-yl)methanone (AM1241), 4-[4-(1,1-dimethylheptyl)-2,6-dimethoxyphenyl]-6,6-dimethyl-bicyclo[3.1.1]hept-2-ene-2-methanol (HU-308), (6aR, 10aR)-9-(hydroxymethyl)-6,6-dimethyl-3-(2-methyloctan-2-yl)-6a, 7,10,10a-tetrahydrobenzo[c]chromen-1-ol (HU-210), (2R,4R,4aR,6S,8aS)-6-(Hydroxymethyl)-4-[2-hydroxy-4-(2-methyl-2-octanyl)phenyl]decahydro-2-naphthalenol (CP55244), 2-[(1 S,3R)-3-hydroxycyclohexyl]-5-(2-methyloctan-2-yl)phenol (CP47497), (11R)-2-Methyl-11-[(morpholin-4-yl)methyl]-3-(naphthalene-1-carbonyl)-9-oxa-1-azatricyclo[6.3.1.0]dodeca-2,4(12), 5,7-tetraene (R-(+)-WIN55212), (2-Methyl-1-propyl-1H-indol-3-yl)-1-naphthalenylmethanone (JWH-015), 1-(2,3-Dichlorobenzoyl)-5-methoxy-2-methyl-3-[2-(4-morpholinyl)ethyl]-1H-indole (L-768242), and any combination thereof.

In some instances, the one or more cannabinoid compounds may be a synthetic eicosanoid selected from the group consisting of methanandamide (R and S isomers), arachidonyl-2-chloroethylamide (ACEA), arachidonylcyclopropylamide (ACPA), and any combination thereof. In some cases, the one or more cannabinoid compounds comprises desacetyl-L-nantradol or 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide] (VYR206).

In some instances, the one or more cannabinoid compounds may be a natural cannabinoid compound. In some cases, the one or more natural cannabinoid compounds comprises a flavonoid or a terpenoid. In other instances, the one or more natural cannabinoid compounds comprises a phytogenic cannabinoid selected from the group consisting of flavonoids, terpenoids, Nabiximols, Cannador, cannabidiol (CBD), cannabinol (CBN), cannabigerol, tetrahydrocannabivarin, cannabichromene, Δ⁸-THC, Δ⁹-tetrahydrocannabinol (Δ⁹-THC), and any combination thereof. In still other instances, the one or more natural cannabinoid compounds comprises an endocannabinoid compound selected from the group consisting of N-arachidonoylethanolamine, (AEA) or anandamide, 2-arachidonoylglycerol (2-AG), noladin ether, virodhamine, N-arachidonylodopamine (NADA), and any combination thereof.

The presently disclosed kits may be used for the diagnostic imaging of an infection site in a subject having an infectious disease. In particular, the present disclosure provides a method of diagnosing an infectious disease in a subject in need thereof. The method may include providing a kit according to the present disclosure and causing the contact of the precursor composition with the label composition to form an image probe composition. The method may further include administering the image probe composition to the subject and performing an imaging technique on the subject or a portion thereof. The imaging technique may be any imaging technique capable of detecting one or more signals from the image probe composition. For example, the imaging technique may be selected from the group consisting of positron emission tomography (PET), computed tomography (CT), single photon emission computed tomography (SPECT), magnetic resonance imaging (MRI), near-infrared (NIR), optical imaging, optoacoustic imaging, ultrasound, and any combination thereof.

The presently disclosed kits may also be used in methods of determining the stage of progression of an infectious disease in a subject as well as to monitor an infectious disease in a subject in need thereof. Similar methods may be used to utilize the presently disclosed kits to treat an infectious disease in a subject as well as image a subject having an infectious disease.

In at least some instances, the infectious disease is a viral infection. For instance, the infectious disease may be a respiratory viral infection selected from the group consisting of human influenza, the common cold, Middle East respiratory syndrome (MERS), severe acute respiratory syndrome coronavirus (SARS), and COVID-19. The infectious disease may also be caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OC43), human coronavirus HKU1 (HCoV HKU1), and human coronavirus 229E (HCoV-229E).

The present disclosure also provides a drug delivery system comprising a kit for delivering a pharmaceutically effective amount of a pharmaceutical composition to a subject in need thereof. In some instances, the drug delivery system may deliver a dual therapeutic intervention agent.

As used herein, the term “conjugate,” in all its forms, refers to a compound formed by the joining of two or more chemical compounds. The term “pharmaceutically acceptable derivative,” as used herein, refers to and includes any pharmaceutically acceptable salt, pro-drug, metabolite, ester, ether, hydrate, polymorph, solvate, complex, and adduct of a compound described herein, which, upon administration to a subject, is capable of providing (directly or indirectly) the active ingredient. For example, the term “a pharmaceutically acceptable derivative” of compounds described herein includes all derivatives of the compounds described herein (such as salts, pro-drugs, metabolites, esters, ethers, hydrates, polymorphs, solvates, complexes, and adducts) which, upon administration to a subject, are capable of providing (directly or indirectly) the compounds described herein. As used herein, the term “pharmaceutically acceptable salt” refers to those salts, which retain the biological effectiveness and properties of the parent compound. Unless otherwise indicated, a pharmaceutically acceptable salt includes salts of acidic or basic groups, which may be present in the compounds of the formulae disclosed herein. The present disclosure also provides certain processes, as examples, for the preparation of the above pharmaceutically acceptable salts, their derivatives, their analogs, their tautomeric forms, their stereoisomers, their polymorphs, and pharmaceutical compositions containing them.

Certain embodiments of the present disclosure relate to pharmaceutically acceptable salts formed by the compounds described herein, or their derivatives, their analogs, their tautomeric forms, their stereoisomers, their polymorphs and pharmaceutically acceptable compositions containing them. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfate, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

In certain embodiments the predetermined quantity of a chelator-nucleoside analog conjugate may be present in dosing amounts to treat a neurologic disorder or cancer as a therapeutic intervention. Combinations containing the label-chelator-nucleoside analog conjugates combine imaging with the therapeutic intervention and can image in real time the uptake and activity of N4 conjugated nucleoside analog, which is essential to select the individual patient with the targeted dysfunctional pathway (right disease) and to assess optimal dosage (right dose). This approach allows for visually seeing the composition located at the tissue site and determining the actual dose of uptake to that site for that patient. This platform allows one to evaluate: (a) if dosing is the cause of the adverse event; (b) if bioavailability is the cause or (c) if there is a limited uptake and/or bio-distribution. In keeping with these parameters, the embodiments serve to dissect effects that are patient dependent, particularly if one of these effects are genetic, epigenetic, or exhibit allelic variations associated to the individual's ECS.

The effective amount of a compound is determined based on several factors, such as age and weight of the patient, severity of the disease, other co-existing factors. The effective amount of a compound includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

The following descriptions of methods, compositions, and results obtained using them are provided merely as illustrative examples. Descriptions of the methods are not intended to require or imply that the steps of the various embodiments must be performed in the order presented. The steps in the foregoing embodiments may be performed in any order. Words such as “then” are not intended to limit the order of the steps; these words are simply used to guide the reader through the description of the methods. Many of the operations may be performed in parallel or concurrently. In addition, the order of the operations may be re-arranged. Various modifications to these embodiments will be readily apparent based on the description provided here, and the generic principles defined here may be applied to other embodiments without departing from the scope of the disclosure.

Further modifications and alternative embodiments of various aspects of the compositions and methods disclosed here will be apparent in view of this description. Accordingly, this description is to be construed as illustrative only and is for the purpose of teaching those skilled in the art the general manner of carrying out the embodiments. It is to be understood that the forms of the embodiments shown and described here are to be taken as examples of embodiments. Elements and materials may be substituted for those illustrated and described here, parts and processes may be reversed or omitted, and certain features of the embodiments may be utilized independently, all as would be apparent after having the benefit of this description of the embodiments. Changes may be made in the elements described here without departing from the scope of the embodiments as described in the following claims.

### EXAMPLES

### Example 1— Synthesis of the Compound According to Formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide

The guanine nucleoside analog compound according to Formula I,

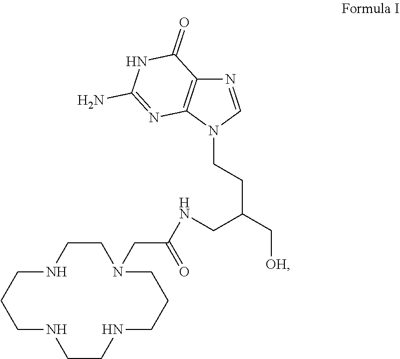 [SMILES: Nc1nc2c(ncn2CCC(CO)CNC(=O)CN2CCCNCCNCCCNCC2)c(=O)[nH]1]

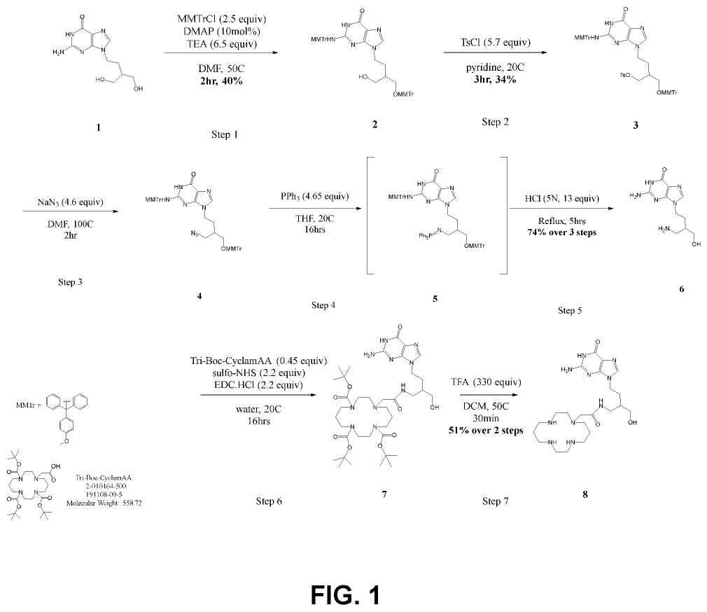

may be synthesized in several ways. FIG. 1 depicts an example process for the synthesis of the compound according to Formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide. As shown in FIG. 1, Compound 1 (penciclovir, 50.0 g, 1 Eq, 197 mmol) may be charged in a 1 L flask with a mechanical stirrer, thermocouple, and nitrogen inlet, followed by the addition of DMSO (300 mL, 60093) (dried over 4A MS) and followed by the addition of triethylamine (44.0 g, 60.5 mL, 2.2 Eq, 434 mmol).

The mixture may be stirred for 10 min to give a white suspension. The stirring may then be increased to vigorous (400-500 RPM). MMTrCl (122 g, 2.0 Eq, 395 mmol) may then be added as a solid while maintaining temperature at 20-25° C. over 20 minutes. An ice bath is then used to periodically lower the reaction temperature. Following the addition, the reaction mixture is a thick brown-black solution. After 4 hrs, the reaction mixture is poured into a mixture of 1.5 L DCM and 1 L water. The reaction mixture is then stirred for 5-10 minutes and let settle for 1 hr before separating the layers. The organic layer is diluted with 1 L water, stirred for 5-10 minutes and let settle for 1 hr. After 1 hr, the mixture is filtered and the solids are discarded. The layers are separated and the organic layer is diluted with 1 L water, stirred for 5-10 minutes and let settle for 1 hr. The layers are separated and after 18 hr aging, the organic layer is filtered and the solids discarded. The organic layer is then dried over sodium sulfate (75 g) and filtered and evaporated filtrate on rotavap (50 mBar, 35 C). Dried briefly under direct vacuum to give 150 g crude solids. Purified by flash chromatography on a 1.5 kg Biotage SNAP Ultra (25 uM) cartridge. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

Compound 2 (450 mg, 1 Eq, 564 μmol) was dissolved in Pyridine (7.5 mL) in a reaction vial with stir bar, thermocouple. Then p-toluenesulfonyl chloride (613 mg, 5.7 Eq, 3.21 mmol) was added over 10 min (9:40 AM-9:50 AM). Color darkens somewhat, very mild exotherm. Temperature remains between 20-22 C. After 3.5 hrs, diluted reaction mixture with EtOAc (20 mL) and water (10 mL). Wash organic with a further 2×10 mL water. Dried organic layer over Na2SO4 (500 mg-1 g), evaporated to dryness. Azeotroped 2×10 mL toluene. Then azeodry 1×10 mL MeCN to yield a yellow solid. Silica chromatography (14×) using 10 g cartridge, Biotage SNAP ultra. Reaction/column monitoring at 254 nm with lambda all detection. solvent. Dissolve in 1 mL EtOAc, liquid loading. Rinse 2 mL 65% EtOAc/heptane. MPA: hept. MPB: EtOAc. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 1 L RBF with stir bar, thermocouple, nitrogen inlet dissolved Compound 3 (53.7 g, 1 Eq) in anhydrous DMF (537 mL, stored over 4A MS) then added sodium azide (5.13 g, 1.4 Eq). Heated to 50 C. After heating for 24 hrs, cooled reaction to room temperature. partitioned mixture between EtOAc (1.5 L) and water (1.5 L). Let settle 1 hr, then split layers. Wash organic 2×1.5 L water further, allowing mixture to settle for 1 hr each time and discarding the rag layer. Dried organic layer over sodium sulfate (57 g). Evaporated on rotavap (35 C, 50 mBar) and dried briefly under direct vacuum to give Compound 4 as a white semisolid, 38.9 g, 75% yield. The resulting compound was analyzed by proton nuclear magnetic resonance CH NMR), carbon-13 nuclear magnetic resonance (¹³C NMR), and high-resolution mass spectrometry (HRMS) by electrospray ionization (ESI).

In a 50 mL RBF with stir bar, condenser, thermocouple, heating mantle, charge Compound 4 (1.00 g, 1 Eq) then THF (15 mL) and water (1.5 mL). Triphenylphosphine (344 mg, 1.2 Eq) was added, and the mixture was heated to 65 C. After 4 hrs, cool reaction to 25 C, add hydrochloric acid (216 mg, 0.18 mL, 2 Eq). The mixture was heated to 65 C. After 3 hrs, cool to room temperature and filter thru 0.2 uM frit. Separated colorless lower layer, transferred to RBF and evaporated to white residue. Dried in vacuum oven (20 C, −29inHg) overnight to give Compound 6 as a white solid (351 mg). A qNMR experiment indicates that the material is 62% potent, with the remainder of mass being water (78% adjusted yield). The resulting compound was analyzed by proton nuclear magnetic resonance CH NMR), carbon-13 nuclear magnetic resonance (¹³C NMR), and high-resolution mass spectrometry (HRMS) by electrospray ionization (ESI).

In a 100 mL RBF, dilute Compound 6 aqueous solution (35.91 g, 29.8 wt %) with 24 g water. Basify to pH 8.1 with 4M NaOH. After 1.5 hrs, add 2 g of celite and filter suspension. Dry solids overnight at ambient temperature (−29inHg) to give Compound 6 as a white solid. Solids were dissolved in 550 mL 20% DMSO/MeOH and filtered. Filtrate was evaporated on a rotavap (40 C, 50 mBar) and then under direct vacuum to give Compound 6 solution in DMSO (41.61 g, 12.6 wt %). Compound 6 DMSO solution (41.61 g, 12.6 wt %) was further diluted with anhydrous DMSO (73 mL) and then anhydrous DMF (212 mL). Added stir bar, nitrogen inlet, thermocouple. Added TriBocCyclamAA (12.9 g, 1.1 Eq) then DMAP (5.13 g, 2.0 Eq) and stirred until mostly dissolved. Then, added EDC·HCl (8.1 g, 2.0 Eq) in a single portion at 20 C. After 24 hrs, the reaction mixture was diluted with DCM (815 mL), 160 mL water, and 650 mL sat. sodium sulfate. The pH of the aqueous layer was adjusted from 8 to 4 using 6M HCl (˜4.5 mL). The biphase was allowed to settle for 1 hr, then the layers were separated. The organic was washed 4× further with 160 mL water, 650 mL sat. sodium sulfate, maintaining the aqueous pH between 4-5 using 6M HCl. The organic layer was dried over sodium sulfate (27 g) and filtered. The sodium sulfate cake was rinsed with 150 mL DCM and the filtrate evaporated on a rotavap (50 mBar, 40 C) then under direct vacuum (−29inHg) to give 31.34 g pale yellow oil (49.1 wt %, 15.4 g intermediate 6, 92% yield). The resulting compound was analyzed by proton nuclear magnetic resonance CH NMR), carbon-13 nuclear magnetic resonance (¹³C NMR), and high-resolution mass spectrometry (HRMS) by electrospray ionization (ESI).

As further shown in FIG. 1, Compound 7 is dissolved (13.2 g, 1 Eq) in DCM (190 mL) and MeCN (20 mL) in 1 L flask with stir bar. Add triethylsilane (19.9 mL, 7.5 Eq) then cool to OC. Add trifluoroacetic acid (51.3 mL, 40 Eq) maintaining temperature <10 C. Following addition warm to room temperature. After 23 hr, charge additional trifluoroacetic acid (12.5 mL, 10 Eq). After 24 hrs, dilute mixture with 40 mL water, stir for 1.5 hr, then let settle for 15 min. Collect faint purple, hazy aqueous lower layer into 1 L RBF. Extract organic additional portion 40 mL water, combine colorless upper aqueous layer with previous aqueous extract. Adjust pH to 8.4 with 4M NaOH, maintaining temperature <35 C. Strip on rotavap (40 C, 50 mBar) then freeze dry overnight to give 180 g crude as an aqueous solution. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

### Example 2—Synthesis of the Compound According to Formula II, N-(9-(4-amino-3-(hydroxymethyl)butyl)-6-oxo-6,9-dihydro-1H-purin-2-yl)-2-(1,4,8,11-tetraazacyclotetradecan-1-yl)acetamide

Synthesis affords the constitutional isomer nucleoside analog compound according to Formula II,

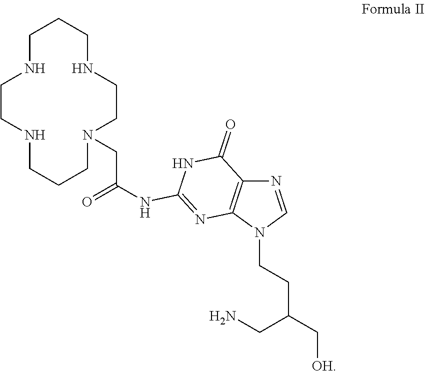 [SMILES: NCC(CO)CCn1cnc2c(=O)[nH]c(NC(=O)CN3CCCNCCNCCCNCC3)nc21]

### Example 3—Synthesis of the Compound According to Formula III, N-(9-(4-(2-(1,4,8,11-tetraazacyclotetradecan-1-yl)acetamido-3-(hydroxymethyl)butyl)-6-oxo-6,9-dihydro-1H -purin-2-yl)-2-(1,4,8,11-tetraazacyclotetradecan-1-yl)acetamide

Synthesis affords the dicyclam product nucleoside analog compound according to Formula III,

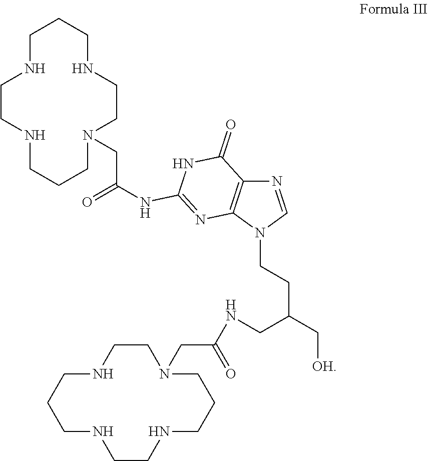 [SMILES: O=C(CN1CCCNCCNCCCNCC1)NCC(CO)CCn1cnc2c(=O)[nH]c(NC(=O)CN3CCCNCCNCCCNCC3)nc21]

### Example 4—Synthesis of the Compound According to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide]

The nucleoside analog compound according to Formula IV,

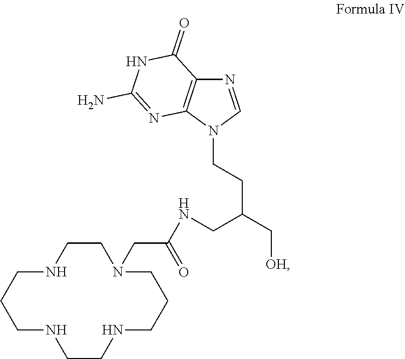 [SMILES: Nc1nc2c(ncn2CCC(CO)CNC(=O)CN2CCCNCCNCCCNCC2)c(=O)[nH]1]

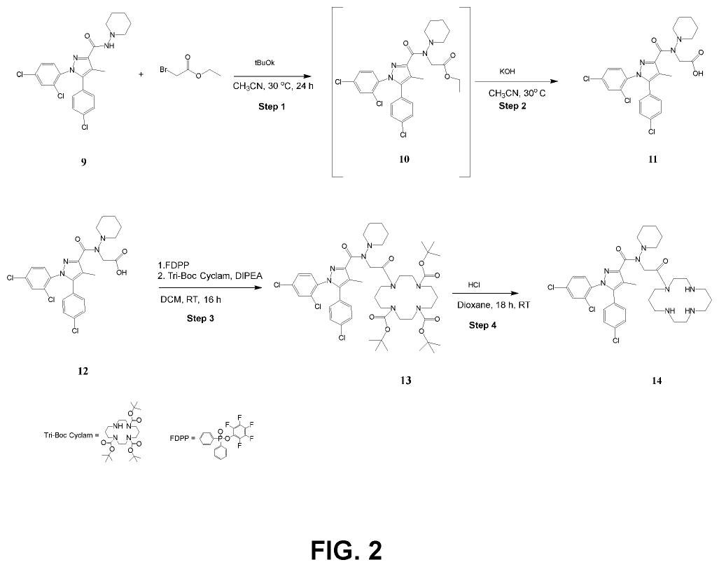

may be synthesized in several ways. FIG. 2 depicts an example process for the synthesis of the compound according to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].

### Statements of the Disclosure:

Statement 1: A kit for preparing an imaging probe composition suitable for administration to a subject in need thereof, the imaging probe composition comprising a conjugate of a nucleoside analog, a chelator, and a label, the kit comprising: a first sealed container comprising a predetermined quantity of a precursor composition, the precursor composition comprising a conjugate of a nucleoside analog and a chelator; and a second sealed container comprising a predetermined quantity of a label composition, the label composition comprising at least one imaging agent or at least one radionuclide label; wherein during use and prior to administration to the subject, the precursor composition is contacted with the label composition to form the imaging probe composition.
Statement 2: The kit according to Statement 1, wherein during use and prior to administration to the subject, the precursor composition in the first sealed container is contacted with the label composition in the second sealed container to form the imaging probe composition.
Statement 3: The kit according to Statement 1, wherein the kit further comprises a third sealed container comprising a predetermined quantity of a cannabinoid composition comprising one or more cannabinoid compounds, wherein the cannabinoid composition and the precursor composition are configured or operable such that when the precursor composition is contacted with the cannabinoid composition a cannabinoid-chelator-nucleoside analog conjugate is formed.
Statement 4: The kit according to Statement 3, wherein during use the precursor composition in the first sealed container is first contacted with the cannabinoid composition in the third sealed container to form the cannabinoid-chelator-nucleoside analog conjugate; and then subsequently the cannabinoid-chelator-nucleoside analog conjugate is contacted with the label composition in the second sealed container to form the imaging probe composition comprising one or more cannabinoid compounds.
Statement 5: The kit according to Statement 1, wherein the conjugate of a nucleoside analog and a chelator comprises one or more cannabinoid compounds such that the conjugate of a nucleoside analog and a chelator is a cannabinoid-chelator-nucleoside analog conjugate.
Statement 6: The kit according to any one of Statements 1-5, wherein at least one of the first sealed container and the second sealed container further comprises a predetermined quantity of a reducing agent, wherein the predetermined quantity of the reducing agent is sufficient to label the conjugate of a nucleoside analog and a chelator with the imaging agent or radionuclide label to form the imaging probe composition.
Statement 7: The kit according to any one of Statements 3-6, wherein the one or more cannabinoid compounds is one or more synthetic cannabinoid compounds.
Statement 8: The kit according to Statement 7, wherein the one or more synthetic cannabinoid compounds comprises a cannabinoid receptor agonist or a cannabinoid receptor antagonist.
Statement 9: The kit according to Statement 8, wherein the cannabinoid receptor is cannabinoid receptor subtype CB₁ or a cannabinoid receptor subtype CB₂.
Statement 10: The kit according to Statement 8, wherein the cannabinoid receptor is a non-CB₁ and a non-CB₂ receptor.
Statement 11: The kit according to Statement 7, wherein the one or more synthetic cannabinoid compounds comprises one or more compounds selected from the group consisting of N-(piperidin-1-yl)-5-(4-chlorophenyl)-1-(2,4-dichlorophenyl)-4-methyl-1H-pyrazole-3-carboxamidehydrochloride (SR141716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3 carboxamide (AM281), 4-[6-Methoxy-2-(4-methoxyphenyl)benzofuran-3-carbonyl]benzonitrile (LY320135), and any combination thereof.
Statement 12: The kit according to Statement 7, wherein the one or more synthetic cannabinoid compounds comprises 5-(1,1-Dimethylheptyl)-2-[5-hydroxy-2-(3-hydroxypropyl)cyclohexyl]phenol (CP-55940), (6aR, 10aR)-6,6,9-trimethyl-3-pentyl-6a,7,8,10a-tetrahydrobenzo[c]chromen-1-ol (Dronabinol or Marinol), or (6aR,10aR)-1-hydroxy-6,6-dimethyl-3-(2-methyloctan-2-yl)-7,8,10,10a-tetrahydro-6aH-benzo[c]chromen-9-one (Nabilone).
Statement 13: The kit according to Statement 7, wherein the one or more synthetic cannabinoid compounds comprises one or more compounds selected from the group consisting of diarylopyrazole, N-(piperidin-1-yl)-5-(4-chlorophenyl)-1-(2,4-dichlorophenyl)-4-methyl-1H-pyrazole-3-carboxamidehydrochloride (SR141716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3carboxamide (AM281), 4-[6-Methoxy-2-(4-methoxyphenyl)benzofuran-3-carbonyl]benzonitrile (LY320135), 5-(1,1-Dimethylheptyl)-2-[5-hydroxy-2-(3-hydroxypropyl)cyclohexyl]phenol (CP-55940), (6aR,10aR)-6,6,9-trimethyl-3-pentyl-6a,7,8,10a-tetrahydrobenzo[c]chromen-1-ol (Dronabinol or Marinol), (6aR,10aR)-1-hydroxy-6,6-dimethyl-3-(2-methyloctan-2-yl)-7,8,10,10a-tetrahydro-6aH-benzo[c]chromen-9-one (Nabilone), an aminoalkylindole, (2-iodo-5-nitrophenyl)-(1-(1-methylpiperidin-2-ylmethyl)-1H-indol-3-yl)methanone (AM1241), 4-[4-(1,1-dimethylheptyl)-2,6-dimethoxyphenyl]-6,6-dimethyl-bicyclo[3.1.1]hept-2-ene-2-methanol (HU-308), (6aR,10aR)-9-(hydroxymethyl)-6,6-dimethyl-3-(2-methyloctan-2-yl)-6a,7,10,10a-tetrahydrobenzo[c]chromen-1-ol (HU-210), (2R,4R,4aR,6 S, 8a5)-6-(Hydroxymethyl)-4-[2-hydroxy-4-(2-methyl-2-octanyl)phenyl]decahydro-2-naphthalenol (CP55244), 2-[(1S,3R)-3-hydroxycyclohexyl]-5-(2-methyloctan-2-yl)phenol (CP47497), (11R)-2-Methyl-11-[(morpholin-4-yl)methyl]-3-(naphthalene-1-carbonyl)-9-oxa-1-azatricyclo[6.3.1.0]dodeca-2,4(12),5,7-tetraene (R-(+)-WIN55212), (2-Methyl-1-propyl-1H-indol-3-yl)-1-naphthalenylmethanone (JWH-015), 1-(2,3-Dichlorobenzoyl)-5-methoxy-2-methyl-3-[2-(4-morpholinyl)ethyl]-1H-indole (L-768242), and any combination thereof.
Statement 14: The kit according to Statement 7, wherein the one or more synthetic cannabinoid compounds comprises a synthetic eicosanoid selected from the group consisting of methanandamide (R and S isomers), arachidonyl-2-chloroethylamide (ACEA), arachidonylcyclopropylamide (ACPA), and any combination thereof.
Statement 15: The kit according to Statement 7, wherein the one or more synthetic cannabinoid compounds comprises desacetyl-L-nantradol or 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide] (VYR206).
Statement 16: The kit according to any one of Statements 3-6, wherein the one or more cannabinoid compounds is one or more natural cannabinoid compounds.
Statement 17: The kit according to Statement 16, wherein the one or more natural cannabinoid compounds comprises a cannabinoid receptor agonist or a cannabinoid receptor antagonist.
Statement 18: The kit according to Statement 17, wherein the cannabinoid receptor is cannabinoid receptor subtype CB₁ or a cannabinoid receptor subtype CB₂.
Statement 19: The kit according to Statement 17, wherein the cannabinoid receptor is a non-CB₁ and a non-CB₂ receptor.
Statement 20: The kit according to Statement 16, wherein the one or more natural cannabinoid compounds comprises a flavonoid or a terpenoid.
Statement 21: The kit according to Statement 16, wherein the one or more natural cannabinoid compounds comprises a phytogenic cannabinoid selected from the group consisting of flavonoids, terpenoids, Nabiximols, Cannador, cannabidiol (CBD), cannabinol (CBN), cannabigerol, tetrahydrocannabivarin, cannabichromene, Δ⁸-THC, Δ⁹-tetrahydrocannabinol (Δ⁹-THC), and any combination thereof.
Statement 22: The kit according to Statement 16, wherein the one or more natural cannabinoid compounds comprises an endocannabinoid compound selected from the group consisting of N-arachidonoylethanolamine, (AEA) or anandamide, 2-arachidonoylglycerol (2-AG), noladin ether, virodhamine, N-arachidonylodopamine (NADA), and any combination thereof.
Statement 23: The kit according to any one of Statements 1-22, wherein the at least one radionuclide label comprises a radionuclide metal ion.
Statement 24: The kit according to any one of Statements 6-23, wherein the reducing agent comprises tin (II) chloride.
Statement 25: The kit according to any one of Statements 3-24, wherein at least one of the first sealed container and the third sealed container further comprises a buffer solution.
Statement 26: The kit according to any one of Statements 1-25, wherein the first sealed container and the precursor composition comprises a reducing agent and a buffer solution.
Statement 27: The kit according to Statement 25 or Statement 26, wherein the buffer solution is a phosphate buffer solution, the phosphate buffer solution in sufficient quantity to stabilize the conjugate of a nucleoside analog and a chelator.
Statement 28: The kit according to Statement 27, wherein the phosphate buffer solution is an aqueous solution comprising at least one selected from the group consisting of monosodium phosphate, disodium phosphate, and any combination thereof.
Statement 29: The kit according to any one of Statements 1-28, wherein the first sealed container and the precursor composition comprises an antioxidant, the antioxidant in sufficient quantity to prevent oxidation of the chelator moiety in the conjugate of a nucleoside analog and a chelator.
Statement 30: The kit according to Statement 29, wherein the antioxidant is vitamin C (ascorbic acid).
Statement 31: The kit according to Statement 29, wherein the antioxidant is selected from the group consisting of tocopherol, pyridoxine, thiamine, butylated hydroxyl toluene, sodium edetate, rutin, vitamin C (ascorbic acid), and any combination thereof.
Statement 32: The kit according to any one of Statements 1-31, wherein the first sealed container and the precursor composition comprises a stabilizer, the stabilizer in sufficient quantity to prevent degradation and enhance shelf life or storage life of the chelator moiety in the conjugate of a nucleoside analog and a chelator.
Statement 33: The kit according to Statement 32, wherein the stabilizer is mannitol.
Statement 34: The kit according to Statement 32, wherein the stabilizer is selected from the group consisting of glucose, lactose, maltose, xylose, sorbitol, cellulose, carboxymethylcellulose sodium, and any combination thereof.
Statement 35: The kit according to Statement 32, wherein the stabilizer is a sugar or bulking agent, the sugar selected from the group consisting of simple sugars, complex chain sugars, sugar alcohols, and any combination or salt thereof.
Statement 36: The kit according to any one of Statements 1-35, wherein at least one of the first sealed container, the second sealed container, and the third sealed container, and/or the respective compositions contained therein, further comprises a pharmaceutically acceptable salt.
Statement 37: The kit according to any one of Statements 1-36, wherein at least one of the first sealed container, the second sealed container, and the third sealed container, and/or the respective compositions contained therein, further comprises a preservative.
Statement 38: The kit according to any one of Statements 1-37, wherein the precursor composition is in liquid, frozen, dry, or lyophilized form.
Statement 39: The kit according to any one of Statements 1-38, wherein the imaging agent composition is in liquid, frozen, dry, or lyophilized form.
Statement 40: The kit according to any one of Statements 1-39, wherein the label composition is in liquid, frozen, dry, or lyophilized form.
Statement 41: The kit according to any one of Statements 1-40, wherein the nucleoside analog is a guanine analog.
Statement 42: The kit according to any one of Statements 1-40, wherein the nucleoside analog is a cell replication check point ligand.
Statement 43: The kit according to any one of Statements 1-40, wherein the nucleoside analog is a synthetic analog.
Statement 44: The kit according to any one of Statements 1-40, wherein the nucleoside analog is a natural analog.
Statement 45: The kit according to any one of Statements 1-40, wherein the nucleoside analog is guanine.
Statement 46: The kit according to any one of Statements 1-40, wherein the nucleoside analog is selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof.
Statement 47: The kit according to any one of Statements 1-40, wherein the nucleoside analog is arabinosyl nucleoside.
Statement 48: The kit according to any one of Statements 1-47, wherein the chelator is an aminated chelator.
Statement 49: The kit according to any one of Statements 1-47, wherein the chelator is an acid chelator.
Statement 50: The kit according to any one of Statements 1-47, wherein the chelator is cyclam.
Statement 51: The kit according to any one of Statements 1-47, wherein the chelator is a N4 chelator or ligand.
Statement 52: The kit according to any one of Statements 1-47, wherein the chelator is an animated chelator.
Statement 53: The kit according to any one of Statements 1-47, wherein the chelator is 6-carboxy-1,4,8,11-tetraazaundecane.
Statement 54: The kit according to any one of Statements 1-47, wherein the chelator is 1,4,8,11-tetraazabicyclohexadecane.
Statement 55: The kit according to any one of Statements 1-54, wherein the radionuclide label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.
Statement 56: The kit according to any one of Statements 1-55, wherein the radionuclide label is configured to facilitate contrast-enhanced imaging when administered to a mammalian subject in conjunction with diagnostic imaging.
Statement 57: The kit according to any one of Statements 1-56, wherein the conjugate in the precursor composition comprises N4-guanine (N4amG).
Statement 58: The kit according to any one of Statements 1-56, wherein the conjugate in the precursor composition comprises cyclam-am-guanine.
Statement 59: The kit according to any one of Statements 1-56, wherein the conjugate in the precursor composition comprises N-(4-(2-amino-6-oxo-1,6,-dihydro-9H-purin-9-yl)-2-(hydroxymethyl)butyl)-2-(1,4,8,11-tetraazacyclotetradecan-1-yl)acetamide.
Statement 60: The kit according to any one of Statements 1-56, wherein the conjugate in the precursor composition comprises a conjugate compound having a structure according to Formula I:

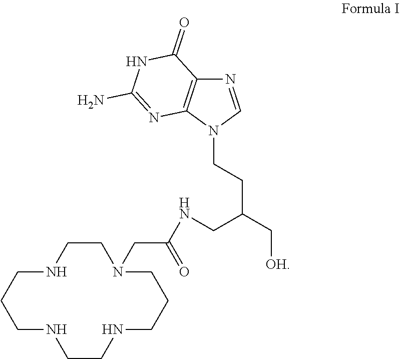 [SMILES: Nc1nc2c(ncn2CCC(CO)CNC(=O)CN2CCCNCCNCCCNCC2)c(=O)[nH]1]

Statement 61: The kit according to any one of Statements 1-56, wherein the conjugate in the precursor composition comprises N-(9-(4-amino-3-(hydroxymethyl)butyl)-6-oxo-6,9-dihydro-1H-purin-2-yl)-2-(1,4,8,11-tetraazacyclotetradecan-1-yl)acetamide.
Statement 62: The kit according to any one of Statements 1-56, wherein the conjugate in the precursor composition comprises a conjugate compound having a structure according to Formula II:

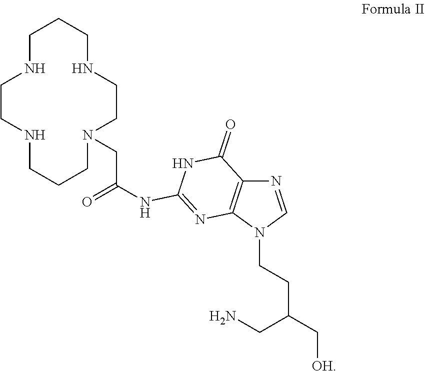 [SMILES: NCC(CO)CCn1cnc2c(=O)[nH]c(NC(=O)CN3CCCNCCNCCCNCC3)nc21]

Statement 63: The kit according to any one of Statements 1-56, wherein the conjugate in the precursor composition comprises N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide.
Statement 64: The kit according to any one of Statements 1-56, wherein the conjugate in the precursor composition comprises a conjugate compound having a structure according to Formula III:

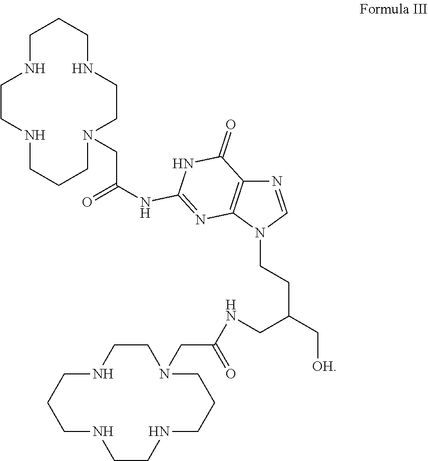 [SMILES: O=C(CN1CCCNCCNCCCNCC1)NCC(CO)CCn1cnc2c(=O)[nH]c(NC(=O)CN3CCCNCCNCCCNCC3)nc21]

Statement 65: The kit according to any one of Statements 1-56, wherein the conjugate in the precursor composition comprises 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].
Statement 66: The kit according to any one of Statements 1-56, wherein the conjugate in the precursor composition comprises a conjugate compound having a structure according to Formula IV:

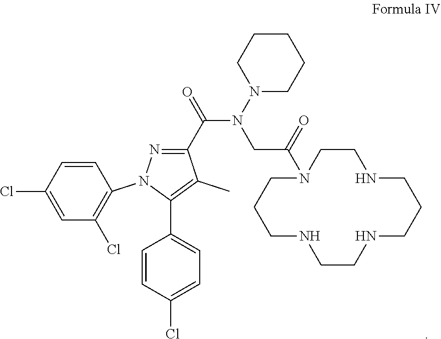 [SMILES: Cc1c(C(=O)N(CC(=O)N2CCCNCCNCCCNCC2)N2CCCCC2)nn(-c2ccc(Cl)cc2Cl)c1-c1ccc(Cl)cc1 ; "."]

Statement 67: A kit for the diagnostic imaging of an infection site in a subject having an infectious disease, the kit comprising a kit according to any one of Statements 1-66.
Statement 68: A method of diagnosing an infectious disease in a subject in need thereof, the method comprising: providing a kit according to any one of Statements 1-66; causing the contact of the precursor composition with the label composition to form an image probe composition; administering the image probe composition to the subject; and performing an imaging technique on the subject or a portion thereof, wherein the imaging technique is capable of detecting one or more signals from the image probe composition.
Statement 69: A method of determining the stage of progression of an infectious disease in a subject in need thereof, the method comprising: providing a kit according to any one of Statements 1-66; causing the contact of the precursor composition with the label composition to form an image probe composition; administering the image probe composition to the subject; and performing an imaging technique on the subject or a portion thereof, wherein the imaging technique is capable of detecting one or more signals from the image probe composition.
Statement 70: A method of monitoring an infectious disease in a subject in need thereof, the method comprising: providing a kit according to any one of Statements 1-66; causing the contact of the precursor composition with the label composition to form an image probe composition; administering the image probe composition to the subject; and performing an imaging technique on the subject or a portion thereof, wherein the imaging technique is capable of detecting one or more signals from the image probe composition.
Statement 71: A method of treating an infectious disease in a subject in need thereof, the method comprising: providing a kit according to any one of Statements 1-66; causing the contact of the precursor composition with the label composition to form an image probe composition; and administering the image probe composition to the subject.
Statement 72: A method of imaging a subject having an infectious disease in a subject, the method comprising: providing a kit according to any one of Statements 1-66; causing the contact of the precursor composition with the label composition to form an image probe composition; administering the image probe composition to the subject; and performing an imaging technique on the subject or a portion thereof, wherein the imaging technique is capable of detecting one or more signals from the image probe composition.
Statement 73: A method according to any one of Statements 68-72, wherein the imaging technique is selected from the group consisting of positron emission tomography (PET), computed tomography (CT), single photon emission computed tomography (SPECT), magnetic resonance imaging (MM), near-infrared (NIR), optical imaging, optoacoustic imaging, ultrasound, and any combination thereof.
Statement 74: A method according to any one of Statements 67-73, wherein the infectious disease is a viral infection.
Statement 75: A method according to any one of Statements 67-73, wherein the infectious disease is a respiratory viral infection selected from the group consisting of human influenza, the common cold, Middle East respiratory syndrome (MERS), severe acute respiratory syndrome coronavirus (SARS), and COVID-19.
Statement 76: A method according to any one of Statements 67-73, wherein the infectious disease is caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OC43), human coronavirus HKU1 (HCoV HKU1), and human coronavirus 229E (HCoV-229E)
Statement 77: A drug delivery system comprising a kit for delivering a pharmaceutically effective amount of a pharmaceutical composition to a subject in need thereof, the kit comprising: a kit according to any one of Statements 1-66; wherein during use the precursor composition is contacted with the label composition to form the pharmaceutical composition.
Statement 78: A method of delivering a predetermined amount of a pharmaceutically acceptable composition to a subject, the method comprising: providing a kit according to any one of Statements 1-66; causing the contact of the precursor composition with the label composition to form the pharmaceutical composition; and administering the pharmaceutical composition to the subject.
Statement 79: A drug delivery system comprising a kit for delivering a dual therapeutic intervention agent to a subject in need thereof, the kit comprising: a kit according to any one of Statements 1-66; wherein during use the precursor composition is contacted with the label composition to form the dual therapeutic intervention agent.
Statement 80: A method of delivering a dual therapeutic intervention agent to a subject, the method comprising: providing a kit according to Statement 79; causing the contact of the precursor composition with the label composition to form the pharmaceutical composition; administering the dual therapeutic intervention agent to the subject.
Statement 81: A kit for preparing a diagnostic agent composition, the diagnostic agent composition comprising a conjugate of a cannabinoid analog, a chelator, and a label, the kit comprising: a kit according to any one of Statements 1-66; wherein during use the precursor composition is contacted with the label composition to form the diagnostic agent composition.
Statement 82: A method of delivering a diagnostic agent comprising a conjugate of a cannabinoid analog to a subject, the method comprising: providing a kit according to Statement 81; causing the contact of the precursor composition with the label composition to form the diagnostic agent; and administering the diagnostic agent to the subject.
Statement 83: The kit or method according to any one of Statements 1-82, wherein the first, second, or third sealed container is a bottle, vial, ampule, or syringe.

1. A kit for preparing an imaging probe composition suitable for administration to a subject in need thereof, the imaging probe composition comprising a conjugate of a nucleoside analog, a chelator, and a label, the kit comprising:
a first sealed container comprising a predetermined quantity of a precursor composition, the precursor composition comprising a conjugate of a nucleoside analog and a chelator; and
a second sealed container comprising a predetermined quantity of a label composition, the label composition comprising at least one an imaging agent or at least one radionuclide label;
wherein during use and prior to administration to the subject, the precursor composition is contacted with the label composition to form the imaging probe composition.

2. The kit according to claim 1, wherein during use and prior to administration to the subject, the precursor composition in the first sealed container is contacted with the label composition in the second sealed container to form the imaging probe composition.

3. The kit according to claim 1, wherein the kit further comprises a third sealed container comprising a predetermined quantity of a cannabinoid composition comprising one or more cannabinoid compounds, wherein the cannabinoid composition and the precursor composition are configured or operable such that when the precursor composition is contacted with the cannabinoid composition a cannabinoid-chelator-nucleoside analog conjugate is formed.

4. The kit according to claim 3, wherein during use the precursor composition in the first sealed container is first contacted with the cannabinoid composition in the third sealed container to form the cannabinoid-chelator-nucleoside analog conjugate; and then subsequently the cannabinoid-chelator-nucleoside analog conjugate is contacted with the label composition in the second sealed container to form the imaging probe composition comprising one or more cannabinoid compounds.

5. The kit according to claim 1, wherein the conjugate of a nucleoside analog and a chelator comprises one or more cannabinoid compounds such that the conjugate of a nucleoside analog and a chelator is a cannabinoid-chelator-nucleoside analog conjugate.

6. The kit according to claim 1, wherein at least one of the first sealed container and the second sealed container further comprises a predetermined quantity of a reducing agent, wherein the predetermined quantity of the reducing agent is sufficient to label the conjugate of a nucleoside analog and a chelator with the imaging agent or radionuclide label to form the imaging probe composition.

7. The kit according to claim 5, wherein the one or more cannabinoid compounds comprises one or more synthetic cannabinoid compounds selected from the group consisting of N-(piperidin-1-yl)-5-(4-chlorophenyl)-1-(2,4-dichlorophenyl)-4-methyl-1H-pyrazole-3-carboxamidehydrochloride (SR141716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3carboxamide (AM281), 4-[6-Methoxy-2-(4-methoxyphenyl)benzofuran-3-carbonyl]benzonitrile (LY320135), and any combination thereof.

8. The kit according to claim 5, wherein the one or more cannabinoid compounds comprises 5-(1,1-Dimethylheptyl)-2-[5-hydroxy-2-(3-hydroxypropyl)cyclohexyl]phenol (CP-55940), (6aR,10aR)-6,6,9-trimethyl-3-pentyl-6a,7,8,10a-tetrahydrobenzo[c]chromen-1-ol (Dronabinol or Marinol), or (6aR,10aR)-1-hydroxy-6,6-dimethyl-3-(2-methyloctan-2-yl)-7,8,10,10a-tetrahydro-6aH-benzo[c]chromen-9-one (Nabilone).

9. The kit according to claim 5, wherein the one or more cannabinoid compounds comprises one or more synthetic cannabinoid compounds selected from the group consisting of diarylopyrazole, N-(piperidin-1-yl)-5-(4-chlorophenyl)-1-(2,4-dichlorophenyl)-4-methyl-1H-pyrazole-3-carboxamidehydrochloride (SR41716A or Rimonabant), N-(piperdin-1-yl)-5-(4-iodophenyl)-1-(2,4-dichlorophenyl)-4-methyl-1H-pyrazole-3-carboxyamide (AM251), N-(morpholin-4-yl)-1-(2,4-dichlorophenyl)-5-(4-iodophenyl)-4-methyl-1H-pyrazole-3carboxamide (AM281), 4-[6-Methoxy-2-(4-methoxyphenyl)benzofuran-3-carbonyl]benzonitrile (LY320135), 5-(1,1-Dimethylheptyl)-2-[5-hydroxy-2-(3-hydroxypropyl)cyclohexyl]phenol (CP-55940), (6aR,10aR)-6,6,9-trimethyl-3-pentyl-6a,7,8,10a-tetrahydrobenzo[c]chromen-1-ol (Dronabinol or Marinol), (6aR,10aR)-1-hydroxy-6,6-dimethyl-3-(2-methyloctan-2-yl)-7,8,10,10a-tetrahydro-6aH-benzo[c]chromen-9-one (Nabilone), an aminoalkylindole, (2-iodo-5-nitrophenyl)-(1-(1-methylpiperidin-2-ylmethyl)-1H-indol-3-yl)methanone (AM1241), 4-[4-(1,1-dimethylheptyl)-2,6-dimethoxyphenyl]-6,6-dimethyl-bicyclo[3.1.1]hept-2-ene-2-methanol (HU-308), (6aR,10aR)-9-(hydroxymethyl)-6,6-dimethyl-3-(2-methyloctan-2-yl)-6a,7,10,10a-tetrahydrobenzo[c]chromen-1-ol (HU-210), (2R,4R,4aR,6S,8aS)-6-(Hydroxymethyl)-4-[2-hydroxy-4-(2-methyl-2-octanyl)phenyl]decahydro-2-naphthalenol (CP55244), 2-[(1S,3R)-3-hydroxycyclohexyl]-5-(2-methyloctan-2-yl)phenol (CP47497), (11R)-2-Methyl-11-[(morpholin-4-yl)methyl]-3-(naphthalene-1-carbonyl)-9-oxa-1-azatricyclo[6.3.1.0]dodeca-2,4(12),5,7-tetraene (R-(+)-WIN55212), (2-Methyl-1-propyl-1H-indol-3-yl)-1-naphthalenylmethanone (JWH-015), 1-(2,3-Dichlorobenzoyl)-5-methoxy-2-methyl-3-[2-(4-morpholinyl)ethyl]-1H-indole (L-768242), and any combination thereof.

10. The kit according to claim 5, wherein the one or more cannabinoid compounds comprises a synthetic eicosanoid selected from the group consisting of methanandamide (R and S isomers), arachidonyl-2-chloroethylamide (ACEA), arachidonylcyclopropylamide (ACPA), and any combination thereof.

11. The kit according to claim 5, wherein the one or more cannabinoid compounds comprises desacetyl-L-nantradol or 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide] (VYR206).

12. The kit according to claim 5, wherein the one or more cannabinoid compounds comprises a flavonoid or a terpenoid.

13. The kit according to claim 5, wherein the one or more cannabinoid compounds comprises a phytogenic cannabinoid selected from the group consisting of flavonoids, terpenoids, Nabiximols, Cannador, cannabidiol (CBD), cannabinol (CBN), cannabigerol, tetrahydrocannabivarin, cannabichromene, Δ8-THC, Δ9-tetrahydrocannabinol (Δ9-THC), and any combination thereof.

14. The kit according to claim 5, wherein the one or more cannabinoid compounds comprises an endocannabinoid compound selected from the group consisting of N-arachidonoylethanolamine, (AEA) or anandamide, 2-arachidonoylglycerol (2-AG), noladin ether, virodhamine, N-arachidonylodopamine (NADA), and any combination thereof.

15. The kit according to claim 1, wherein the first sealed container and the precursor composition comprises a reducing agent and a buffer solution, wherein the buffer solution is a phosphate buffer solution, the phosphate buffer solution in sufficient quantity to stabilize the conjugate of a nucleoside analog and a chelator.

16. The kit according to claim 15, wherein the first sealed container and the precursor composition comprises an antioxidant, the antioxidant in sufficient quantity to prevent oxidation of the chelator moiety in the conjugate of a nucleoside analog and a chelator, wherein the antioxidant is selected from the group consisting of tocopherol, pyridoxine, thiamine, butylated hydroxyl toluene, sodium edetate, rutin, vitamin C (ascorbic acid), and any combination thereof.

17. The kit according to claim 16, wherein the first sealed container and the precursor composition comprises a stabilizer, the stabilizer in sufficient quantity to prevent degradation and enhance shelf life or storage life of the chelator moiety in the conjugate of a nucleoside analog and a chelator. wherein the stabilizer is selected from the group consisting of glucose, lactose, maltose, xylose, sorbitol, cellulose, carboxymethylcellulose sodium, and any combination thereof.

18. The kit according to claim 1, wherein the nucleoside analog is selected from the group consisting of adenine, adenosine, deoxyadenosine, guanine, guanosine, dexoyguanosine, thymine, 5-methyluridine, thymidine, uracile, uridine, deoxyuridine, cytosine, cytidine, deoxycytidine, and any combination thereof.

19. The kit according to claim 18, wherein the chelator is cyclam.

20. The kit according to claim 19, wherein the radionuclide label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.
